(19) United States
(12) Patent Application Publication
(10) Pub. No.: US 2019/0108307 A1
(43) Pub. Date: Apr. 11, 2019

(54) DESIGNER NUTRITIONAL SUPPLEMENT AND ROUTE FOR INSECT TRANSPORT

(71) Applicant: INTERNATIONAL BUSINESS MACHINES CORPORATION, Armonk, NY (US)

(72) Inventors: Pasquale A. CATALANO, Wallkill, NY (US); Andrew G. CRIMMINS, Montrose, NY (US); Robert K. OVERTON, Olivebridge, NY (US); Anuradha RAO, Hopewell Jackson, NY (US)

(21) Appl. No.: 15/729,082

(22) Filed: Oct. 10, 2017

(51) Int. Cl. G06F 19/12; A23K 50/90; A23K 20/142

(52) U.S. Cl. CPC G06F 19/12; G06N 99/005; A23K 20/142; A23K 50/90

(57) ABSTRACT

Embodiments include methods, systems, and computer program products for generating a designer nutrition supplement for insect transport is provided. Aspects include receiving a target location and target commercial activity for an insect. Aspects include determining an arrival time window for the target location. Aspects include receiving a base nutrition supplement formula for the insect. Aspects include determining a commercially-based nutrition modification to optimize the target commercial activity. Aspects include generating a designer nutrition supplement specification based at least in part upon the commercially-based nutrition modification.

### BACKGROUND

The present invention relates to insect transport, and more specifically, to designer nutritional supplements and optimal routes for insect transport.

Insects can be transported via trucks for a variety of commercial activities. For instance, bees play an important role in the successful growth of many types of crops through pollination. It has been estimated that seventy percent of the top human food crops are pollinated by bees. Modern agriculture increasingly relies upon commercial beekeepers to supply bees needed to pollinate crops. In commercial beekeeping operations, hives can be transported by truck to a number of different types of crops over the course of a year. Over the past decade, bee populations have suffered devastating reductions in numbers due to colony collapse and other maladies Maintaining and improving the health of existing bee populations is not only beneficial to the environment but can be crucial to agricultural food production. During transport of bees and other insects, nutritional supplements can be provided.

### SUMMARY

In accordance with one or more embodiments, a computer-implemented method for generating a designer nutritional supplement for insect transport is provided. The method includes receiving, by a processor, a target location and target commercial activity for an insect. The method also includes determining, by the processor, an arrival time window for the target location. The method also includes receiving, by the processor, a base nutrition supplement formula for the insect. The method also includes determining, by the processor, a commercially-based nutrition modification to optimize the target commercial activity. The method also includes generating, by the processor, a designer nutrition supplement specification based at least in part upon the commercially-based nutrition modification.

In accordance with another embodiment, a computer program product for generating a designer nutrition supplement for insect transport is provided. The computer program product includes a computer readable storage medium readable by a processing circuit and storing program instructions for execution by the processing circuit for performing a method. The method includes receiving a target location and target commercial activity for an insect. The method also includes determining an arrival time window for the target location. The method also includes receiving a base nutrition supplement formula for the insect. The method also includes determining a commercially-based nutrition modification to optimize the target commercial activity. The method also includes generating a designer nutrition supplement specification based at least in part upon the commercially-based nutrition modification.

In accordance with a further embodiment, a processing system for generating a designer nutritional supplement for insect transport includes a processor in communication with one or more types of memory. The processor is configured to receive a target location and target commercial activity for an insect. The processor is also configured to determine an arrival time window for the target location. The processor is also configured to receive a base nutrition supplement formula for the insect. The processor is also configured to determine a commercially-based nutrition modification to optimize the target commercial activity. The processor is also configured to generate a designer nutrition supplement specification based at least in part upon the commercially-based nutrition modification.

### BRIEF DESCRIPTION OF THE DRAWINGS

The subject matter of the present invention is particularly pointed out and distinctly claimed in the claims at the conclusion of the specification. The foregoing and other features and advantages of the one or more embodiments described herein are apparent from the following detailed description taken in conjunction with the accompanying drawings in which:

FIG. 1 is block diagram illustrating one example of a processing system for practice of the teachings herein;

FIG. 2 is a block diagram illustrating a system for generating designer nutritional supplements and optimal routes for insect transport according to one or more embodiments of the present invention.

FIG. 3 is a flow diagram illustrating a method for generating a designer nutrition supplement specification according to one or more embodiments of the present invention.

FIG. 4 depicts a flow diagram illustrating an exemplary method for generating an optimal insect route plan according to one or more embodiments of the present invention.

FIG. 5 is a block diagram illustrating a system for generating designer nutritional supplements and optimal routes for insect transport according to one or more embodiments of the present invention.

### DETAILED DESCRIPTION

Insects can be transported to various locations for commercial purposes. For instance, in the agricultural industry, pollinators such as bees can be transported to a series of crops over the course of a year to increase crop production.

Commercial pollinators for instance, can play a crucial role in the production of certain fruits, nuts, and vegetables. To meet increasing demands for high agricultural output, farms increasingly seek to lease pollinators from commercial sources, such as commercial bees, which are transported to their crops at a time within the crop pollination window.

The destination for commercial bee enterprises can span the length of the United States and involve extended periods of time in potentially harsh conditions. In the case of migratory commercial bees, hives can be kept overwinter in a favorable climate, where there is flowering year round, and then loaded onto a truck for extended transport. For instance, after overwintering in Florida, exemplary beehives can be transported to California in February to pollinate almonds and other nut crops such as pistachios. After California, the same hives can be transported by truck to Washington and New York to pollinate apples and other stone fruit, to Maine to pollinate blueberries, Pennsylvania to pollinate squash and pumpkins, and then back to Florida to overwinter.

Conditions that insects such as bees can be exposed to during transport can be harsh, can adversely affect their health and could cause death or colony collapse. For example, commercial bees during transport are exposed to exhaust fumes, temperature fluctuations, dietary imbalances, and other conditions far from natural migratory behavior and environments, which can stress the bee colonies. In addition, the overall health and diet of transported insects can be neglected and is often not considered before or during transport.

Such stressors could be contributing factors to Colony Collapse Disorder (CCD) in commercial beekeeping. CCD is a phenomenon in which bee colonies essentially collapse, in that a majority of worker bees abruptly disappear from the colony although the queen and ample food stores remain in the hive. CCD is believed to be caused by multiple stressors, such as combinations of pesticide exposure, disease, mites and other pests, environmental factors such as temperature, and nutrition and food deficiencies.

Embodiments of the present invention include methods and systems for improved commercial insect transport. Some embodiments of the invention provide systems and methods for designer nutritional supplements for commercial insect transport. In some embodiments of the invention, designer nutritional supplements are based upon environmental factors or commercial activities. In some embodiments of the invention, nutritional supplements are designed by cognitive techniques to promote the overall health of the insects during transport. In some embodiments of the invention, nutritional supplements are designed by cognitive techniques to nutritionally prepare the insects for desired activities at target locations. In some embodiments of the invention commercial migratory insect populations can be intentionally weaned of nutrients, such as certain amino acids, vitamins and minerals, and proteins that will be in excess at commercial destinations to improve health or productivity upon arrival at the destinations. For example, in the case of commercial migratory bees, embodiments of the invention can provide a designer nutritional supplement or supplement plan that contains all essential nutrients needed in a healthy bee diet while selectively and gradually weaning the bees of amino acids that will be present at a target crop they will be required to pollinate shortly before arrival at the target. The withdrawal of specific amino acids can drive the bees to harvest nectar from the target crops that will supplement the nutrition deficiency, thereby driving greater pollination of the target crop.

In some embodiments of the invention, systems and methods provide a route for optimal insect transport based upon a cognitive model using environmental factors, such as weather, and target locations. For example, embodiments of the invention can provide an optimal route for commercial migratory beekeeping operations based upon cognitive models, for instance in which predictive models are used to determine bloom time windows for target crops based upon a variety of factors, such as crop type, location, humidity, temperature and rain levels and a route is generated based upon the bloom time windows and target crop locations.

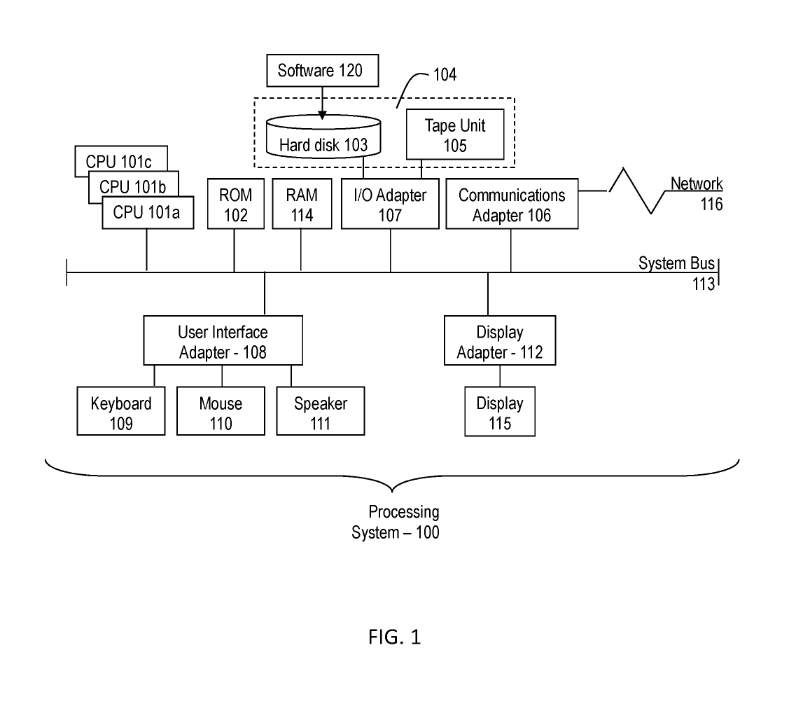

Referring to FIG. 1, there is shown an embodiment of a processing system 100 for implementing the teachings herein. In this embodiment, the system 100 has one or more central processing units (processors) 101a, 101b, 101c, etc. (collectively or generically referred to as processor(s) 101). In one embodiment, each processor 101 may include a reduced instruction set computer (RISC) microprocessor. Processors 101 are coupled to system memory 114 and various other components via a system bus 113. Read only memory (ROM) 102 is coupled to the system bus 113 and may include a basic input/output system (BIOS), which controls certain basic functions of system 100.

FIG. 1 further depicts an input/output (I/O) adapter 107 and a network adapter 106 coupled to the system bus 113. I/O adapter 107 may be a small computer system interface (SCSI) adapter that communicates with a hard disk 103 and/or tape storage drive 105 or any other similar component. I/O adapter 107, hard disk 103, and tape storage device 105 are collectively referred to herein as mass storage 104. Software 120 for execution on the processing system 100 may be stored in mass storage 104. A network adapter 106 interconnects bus 113 with an outside network 116 enabling data processing system 100 to communicate with other such systems. A screen (e.g., a display monitor) 115 is connected to system bus 113 by display adaptor 112, which may include a graphics adapter to improve the performance of graphics intensive applications and a video controller. In one embodiment, adapters 107, 106, and 112 may be connected to one or more I/O busses that are connected to system bus 113 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 113 via user interface adapter 108 and display adapter 112. A keyboard 109, mouse 110, and speaker 111 all interconnected to bus 113 via user interface adapter 108, which may include, for example, a Super I/O chip integrating multiple device adapters into a single integrated circuit.

Thus, as configured in FIG. 1, the system 100 includes processing capability in the form of processors 101, storage capability including system memory 114 and mass storage 104, input means such as keyboard 109 and mouse 110, and output capability including speaker 111 and display 115. In one embodiment, a portion of system memory 114 and mass storage 104 collectively store an operating system such as the AIX® operating system from IBM Corporation to coordinate the functions of the various components shown in FIG. 1.

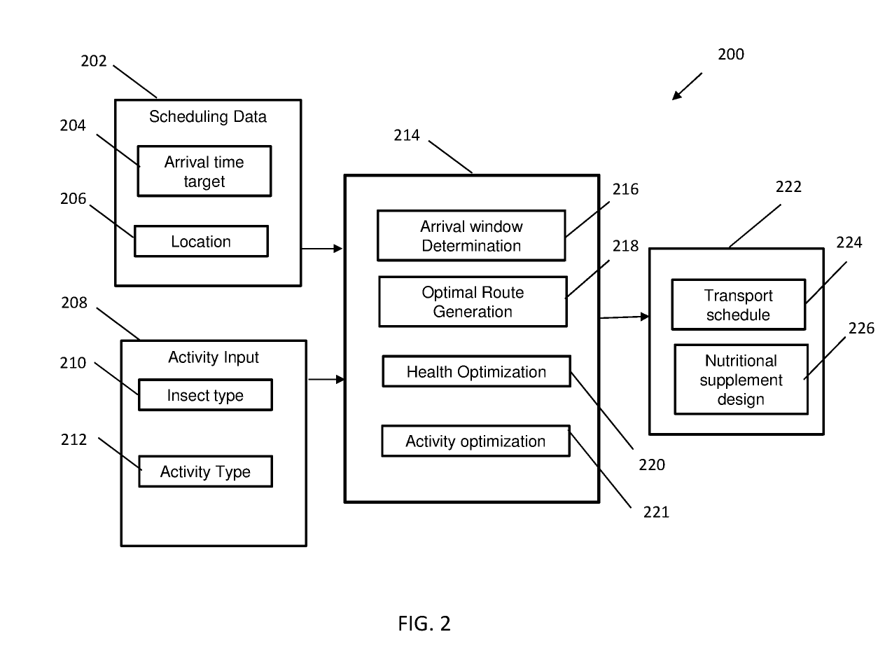

FIG. 2 is a block diagram illustrating a system for generating designer nutritional supplements and optimal routes for insect transport according to one or more embodiments of the present invention. The system 200 can include a scheduling data input 202 and an activity input 208. The scheduling data input 202 can include data relevant to scheduling and planning a route for insect transport, such as an arrival time target 204 and a location 206. The arrival time target 204 can include manual user entries, such as customer preference, and/or calculated or derived arrival times based upon data relevant to the insect, target, and/or target activity. In some embodiments of the invention, an arrival time target 204 can be generated by cognitive learning, for example by machine learning. An activity input 208 can include data relevant to activities involved in the insect transport, such as insect type 210, including for example bees, crickets, butterflies, ladybugs, and activity type 212, such as crop pollination, agricultural pest control, animal feeding activities.

A processing hub 214 can include an arrival window determination module 216, an optimal route generation module 218, a health optimization module 220, and/or an activity optimization module 221.

The arrival window determination module 216 can determine arrival time window for a given location based upon scheduling or activity data. In some embodiments of the invention, the arrival window determination module 216 can generate bloom time windows for a target crop based upon historic bloom time data for the target crop or for crops of the same type as the target crop, past and predicted weather and environmental data, such as temperature, pollen data, humidity, rain levels, pollution levels, geographic location, and/or the type and location of other target crops for the insects.

In some embodiments of the invention, an optimal route generation module 218 generates an optimal route or plurality of routes for the insects based upon one or more of an arrival window and/or arrival time target, locations of targets, insect types, and activity type. For example, an optimal route generation module can plan a route for a commercial beekeeping truck that minimizes road time or maximizes a number of targets while arriving at each target location within a provided or generated bloom time window.

In some embodiments of the invention, a health optimization module 220 is included. The health optimization module 220 can modify a route or nutrition supplement specification to improve insect health based upon the route or environmental factors. For example, a health optimization module 220 can determine if a trip between locations is relatively long and, accordingly, can modify a nutrition supplement specification to include detoxifying agents to improve insect health.

In some embodiments of the invention, an activity optimization module 221 is included. The activity optimization module 221 can modify a nutrition supplement specification to improve the output of a target commercial activity. For example, an activity optimization module 221 can determine a commercially-based nutrition modification to optimize a target commercial activity. For instance, the activity optimization module for a commercial beekeeping operation can determine one or more target amino acids associated with the target crop and generate a designer nutrition supplement specification designed to withdraw the target amino acid from the bee diet within a designated time frame prior to arrival at the target. The designated time frame and the nature of the reduction of target amino acid (gradual versus abrupt) can depend upon the route (for instance, the duration between targets, the presence of multiple targets of the same type, etc.), the nature of the target crop (the level of nutrition provided by the target, the expected stressors at the target crop, such as pollution levels and climate issues), and the nature of the amino acid to be reduced. For example, a weaning time can be, for instance, on the order of 2 days to 2 weeks.

As used herein, a nutrition supplement specification or a nutrition specification can include any directive, formula, recipe, ingredient listing, or other means of communicating the composition of a nutrition supplement including, for instance, the types and amounts of nutritional components, such as amino acids, vitamins, fats, and carbohydrates, and optionally of excipients such as fillers, binders, and preservatives.

The system 200 can include an output interface 222, including for example a transport schedule 224 and/or a nutritional supplement design specification 226.

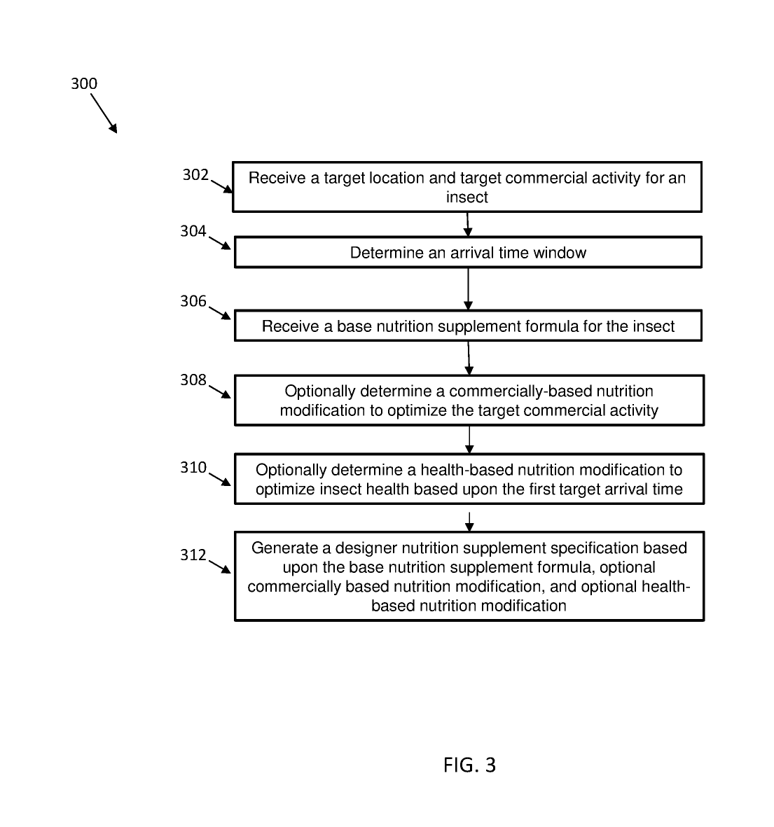

FIG. 3 is a flow diagram illustrating a method 300 for generating a designer nutrition supplement specification according to one or more embodiments of the present invention. The method 300 includes, as illustrated at block 302, receiving a target location and target commercial activity for an insect. The method 300 also includes, as shown at block 304, determining an arrival time window. The arrival time window can be determined in some embodiments by machine learning techniques based at least in part upon the target location and the target commercial activity, and can also include, for example, the location of other targets, stated or calculated bloom times for target crops in the case of agricultural activities, customer demand times, and the like. The method 300 also includes, as shown at block 306, receiving a base nutrition supplement formula for the insect. The method 300 also includes, as shown at block 308, optionally determining a commercially-based nutrition modification to optimize the target commercial activity. The method 300 also includes, as shown at block 310, optionally determining a health-based nutrition modification to optimize insect health based upon the first target arrival time. The method 300 also includes, as shown at block 312, generating a designer nutrition supplement specification based upon the base nutrition supplement formula, optional commercially based nutrition modification, and optional health-based nutrition modification.

Any insect that can be transported in large quantities (for instance greater than 500 insects) to multiple destinations can be included in embodiments of the invention. Exemplary insects include pollinating insects, such as bees, butterflies, wasps, ants, flies, midges, moths, beetles. Other insects can include, for example, insects that provide pest removal and control, such as ladybugs or dragonflies, and insects that provide nutrition for other pets and animals, such as crickets. Base nutrition supplement formulas for insects are known and/or can be readily determined by those skilled in the art and can depend upon the insect type. Target commercial activities can include, for example, crop pollination, agricultural pest control, and animal feeding.

Commercially-based nutrition modifications include modifications to the base nutrition designed to improve a commercial activity. For instance, a pollinating insect can derive a variety of proteins and amino acids from pollen, but the composition of pollen, including the amount and type of amino acids present, varies depending upon the type of crop. Commercially-based nutrition modifications can, for example, withhold a source of material that will be in excess at a target location, for instance when the insect is a pollinator or is sought for pest control, to increase the insect's affinity for the desired target.

Exemplary crops that can be pollinated by bees include, but are not limited to fruits, vegetables, and nuts, including for instance, oranges, almonds, watermelons, cucumbers, apples, avocados, blueberries, and peaches.

Health-based nutrition modification can include modifications to a base nutrition designed to improve health of the insects. For instance, a commercial migratory bee colony may be required to pollinate crops that are deficient in certain amino acids needed to maintain bee health. Health-based nutrition modifications can include supplementation with extra amino acids or vitamins of a type known to be absent in a target crop or supplementation with additional carbohydrates during periods of prolonged natural food source deficits. Health-based nutrition modifications can include the addition of agents to counteract one or more stressors of travel, for instance detoxifying agents can be used in cases and at times where insects are exposed to prolonged travel or exhaust fume exposure or exposure to other pollutants. Exemplary detoxifying agents are known and can include, for instance, cytochrome P450 monooxygenases, glutathione-S-transferases, and carboxylesterases.

A designer nutrition supplement specification can include a listing of nutrients and nutrient amounts that vary over the course of intended travel for a traveling insect population. The designer nutrition supplement specification can include two or more nutrition scheduling windows, wherein each of the nutrition scheduling windows specifies a nutrition supplement formula based upon the route and/or target commercial activity with one or more different nutrients or nutrient amounts. In some embodiments of the invention, the designer nutrition supplement specification includes four or more scheduling windows.

In some embodiments of the invention, determining the commercially-based nutrition modification includes generating a first nutrition scheduling window and a second nutrition scheduling window. In some embodiments of the invention, the first nutrition scheduling window comprises a base nutrition specification comprising a first amount of an amino acid. In some embodiments of the invention, the second nutrition scheduling window comprises a weaning nutrition specification comprising a second amount of the amino acid, wherein the second amount of the amino acid is less than the first amount of the amino acid. The second amount of the amino acid can be, for example, 20%, 50%, or 100% less than the first amount of the amino acid. The first nutrition scheduling window and the second scheduling window can be determined based upon the target location, the location type, the arrival time window, travel time to the location, a desired weaning time in the case of a commercially-based modification relying upon component reduction, and/or any aspects relevant to nutrition modifications, such as the need for detoxifying agents, sugar supplementation, etc.

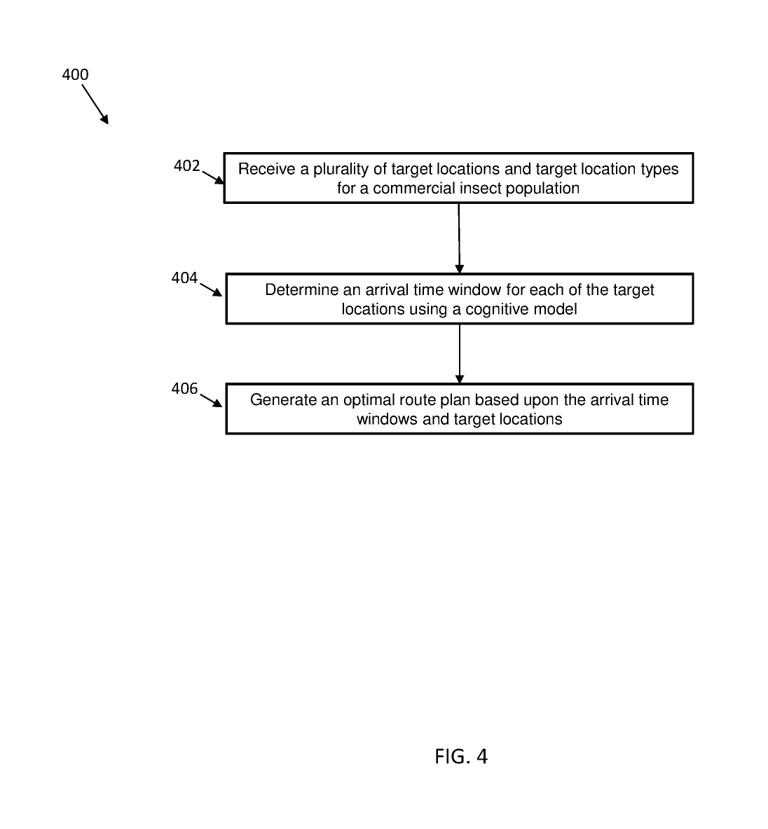

FIG. 4 depicts a flow diagram illustrating an exemplary method 400 for generating an optimal insect route plan according to one or more embodiments of the present invention. The method 400 includes receiving a plurality of target locations and target location types for a commercial insect population, as shown at block 402. The method also includes determining an arrival time window for each of the target locations using a cognitive model, as shown at block 404. The method 400 also includes, as shown at block 406, generating an optimal route plan based upon the arrival time windows and target locations.

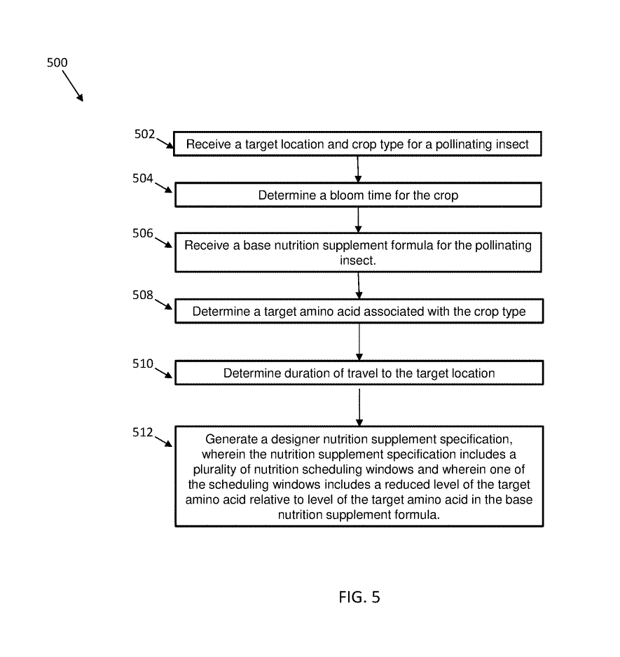

FIG. 5 depicts a flow diagram illustrating an exemplary method 500 for generating a designer nutrition supplement for a pollinating insect, such as a bee. The method 500 includes, as shown at block 502, receiving a target location and crop type for a pollinating insect. The method 500 includes, as shown at block 504, determining a bloom time for the crop, for example by a machine learning method using historic and predicted bloom times and environmental information for the crop. The method 500 also includes, as shown at block 506, receiving a base nutrition supplement formula for the pollinating insect. The method 500 also includes, as shown at block 508, determining a target amino acid associated with the crop type. The target amino acid can include, for example, an essential amino acid for bees (arginine, histidine, isoleucine, leucine, lysing, methionine, phenylalanine, threonine, tryptophan, valine) but that can be reduced in an from the bee diet for a short period of time prior to crop pollination without destroying the colony. The target amino acid can vary and is dependent upon the target crop type and location. Amino acid content of various crops are known and/or can be readily determined by those skilled in the art for example by high performance liquid chromatography (HPLC). The method 500 can also include, as shown at block 510, determining a duration of travel to the target location. The method can also include, as shown at block 512, generating a designer nutrition supplement specification, wherein nutrition supplement specification includes a plurality of nutrition scheduling windows and wherein one of the scheduling windows includes a reduced level of the target amino acid relative to level of the target amino acid in the base nutrition supplement formula.

In some embodiments of the invention, methods include preparing a nutrition supplement according to the designer nutrition supplement specification.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form described. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The flow diagrams depicted herein are just one example. There can be many variations to this diagram or the steps (or operations) described therein without departing from the spirit of embodiments of the invention. For instance, the steps can be performed in a differing order or steps can be added, deleted or modified. All of these variations are considered a part of the claimed invention.

The descriptions of the various embodiments of the present invention have been presented for purposes of illustration, but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

1-7. (canceled)

8. A computer program product for generating a designer nutrition supplement for insect transport, the computer program product comprising:
a computer readable storage medium readable by a processing circuit and storing program instructions for execution by the processing circuit for performing a method comprising:
receiving a target location and target commercial activity for an insect;
determining an arrival time window for the target location;
receiving a base nutrition supplement formula for the insect;
determining a commercially-based nutrition modification to optimize the target commercial activity; and
generating a designer nutrition supplement specification based at least in part upon the commercially-based nutrition modification.

9. The computer program product of claim 8, wherein the insect is a pollinating insect.

10. The computer program product of claim 8, wherein the target commercial activity is crop pollination.

11. The computer program product of claim 8, wherein determining the arrival time window for the target location comprises using a machine learning method.

12. The computer program product of claim 8, wherein determining the commercially-based nutrition modification comprises generating a first nutrition scheduling window and a second nutrition scheduling window, wherein
the first nutrition scheduling window comprises a base nutrition specification comprising a first amount of an amino acid; and
the second nutrition scheduling window comprises a weaning nutrition specification comprising a second amount of the amino acid, wherein the second amount of the amino acid is less than the first amount of the amino acid.

13. The computer program product of claim 8, wherein the method further comprises determining a health-based nutrition modification.

14. The computer program product of claim 13, wherein the designer nutrition supplement specification is based at least in part upon the health-based nutrition modification.

15. A processing system for generating a designer nutritional supplement for insect transport, comprising:
a processor in communication with one or more types of memory, the processor configured to:
receive a target location and target commercial activity for an insect;
determine an arrival time window for the target location;
receive a base nutrition supplement formula for the insect;
determine a commercially-based nutrition modification to optimize the target commercial activity; and
generate a designer nutrition supplement specification based at least in part upon the commercially-based nutrition modification.

16. The processing system of claim 15, wherein the insect is a pollinating insect.

17. The processing system of claim 15, wherein the target commercial activity is crop pollination.

18. The processing system of claim 15, determining, by the processor, the arrival time window for the target location comprises using a machine learning method.

19. The processing system of claim 15, wherein determining the commercially-based nutrition modification comprises generating a first nutrition scheduling window and a second nutrition scheduling window, wherein
the first nutrition scheduling window comprises a base nutrition specification comprising a first amount of an amino acid; and
the second nutrition scheduling window comprises a weaning nutrition specification comprising a second amount of the amino acid, wherein the second amount of the amino acid is less than the first amount of the amino acid.

20. The processing system of claim 15, wherein the processor is configured to determine a health-based nutrition modification.
